(19) United States
(12) Patent Application Publication
(10) Pub. No.: US 2004/0022752 A1
(43) Pub. Date: Feb. 5, 2004

(54) Film-forming cosmetic composition

(76) Inventor: Valerie De La Poterie, Le Chalelet en Brie (FR)

Correspondence Address: FINNEGAN, HENDERSON, FARABOW, GARRETT & DUNNER, LLP, 1300 I STREET, NW, WASHINGTON, DC, 20005, US

(21) Appl. No.: 10/415,137

(22) PCT Filed: Oct. 26, 2001
(86) PCT No.: PCT/FR01/03350

(30) Foreign Application Priority Data
Oct. 27, 2000 (FR) 00/13877

(51) Int. Cl. A61K 7/06; A61K 7/11

(52) U.S. Cl. 424/70.11; 424/70.17

(57) ABSTRACT

The invention concerns a composition comprising, in a physiologically acceptable medium containing an aqueous phase,: an aqueous dispersion of film-forming particles, an aqueous micro-dispersion of particles of a first wax having a melting point not less than 70 ° C., the particles of the first wax having an average size less than 1 μm, and an aqueous dispersion of particles having a melting point less than 70 ° C., the particles of the second wax having an average size not less than 1 μm. The composition provides a make-up resistant to cold water. It also provides the eyelashes good curved shape. The invention is applicable to make-up and care of keratinous matter, in particular mascara.

The present invention relates to a cosmetic composition comprising a film-forming polymer and waxes, which is intended for making up or for cosmetic care of keratin materials such as keratin fibres, for instance the eyelashes, the eyebrows or the hair, and also the skin and the nails, especially of human beings. The invention also relates to a cosmetic care and make-up process for keratin materials.

The composition may be in the form of a mascara, an eyeliner, a product for the lips, a face powder, an eye shadow, a foundation, a make-up product for the body, a concealer, a product for the nails, an antisun composition, a skin-colouring composition or a skincare product. More especially, the invention relates to a mascara.

The term “mascara” means a composition intended to be applied to the eyelashes: it may be a make-up composition for the eyelashes, a make-up base for the eyelashes, a composition to be applied over a mascara, also known as a top coat, or even a cosmetic treatment composition for the eyelashes. The mascara is more particularly intended for human eyelashes, but also for false eyelashes.

Mascara compositions in the form of a wax-in-water emulsion comprising surfactants are known from document WO-A-95/15741. However, the make-up film obtained with these compositions does not show good resistance to cold water and when the film comes into contact with water, during bathing or showering, for example, it disintegrates partially by crumbling away or by spreading around the eye. The crumbling of the film gives rise to a substantial loss of the intensity of the colour of the make-up, thus making it necessary for the consumer to renew the application of the mascara. As regards the spreading of the film, this forms a highly unattractive halo around the made-up area. Tears and perspiration also cause these same drawbacks.

To promote the water resistance of make-up, it is known practice from document U.S. Pat. No. 4,423,031 to use film-forming polymers (especially acrylic polymers) in aqueous dispersion (also known as latices) and waxes.

To obtain a thick deposit of the composition on the eyelashes, that is to say to obtain a loading mascara, the composition generally comprises a waxes-in-water emulsion, the waxes possibly having a melting point ranging from 40° C. to 120° C. The use of waxes having a melting point of greater than or equal to 70° C. in the mascara composition makes it necessary to preheat the aqueous dispersion of film-forming polymer to a temperature above ₈₀° C. However, at these high temperatures, the aqueous polymer dispersion may be destabilized: the polymer particles no longer remain in homogeneous dispersion but instead flocculate, making the composition unusable.

The aim of the present invention is thus to propose a cosmetic composition comprising a film-forming polymer in aqueous dispersion and waxes having a melting point of greater than or equal to 70° C., whose use is compatible with the said film-forming polymer.

An aim of the invention is also to obtain a cosmetic composition which is capable of forming a thick deposit on keratin materials and which is resistant to cold water.

An aim of the invention is also to propose a cosmetic composition which imparts good curling to keratin fibres.

The inventors have discovered that such a composition can be obtained by using a microdispersion of high-melting wax and a dispersion of low-melting wax, combined with an aqueous dispersion of film-forming polymer particles.

After applying the composition to the keratin materials, especially to the eyelashes, the make-up result obtained shows good staying power: the film is indeed resistant to cold water, that is to say to water with a temperature of less than or equal to 30° C., for example during bathing, and/or to tears and/or to perspiration; the film is also resistant to rubbing, for example with fingers or fabrics (handkerchiefs or towels). The composition also makes it possible to obtain a thick deposit on keratin materials. In particular, when the composition is a mascara intended to be applied to keratin fibres, for instance the eyelashes, the said eyelashes are considerably thickened. The mascara is also referred to as being “loading”. Moreover, the composition allows the eyelashes to be curled. Thus, the mascara according to the invention is both loading and curling and shows good staying power.

More specifically, one subject of the invention is a composition comprising, in a physiologically acceptable medium containing an aqueous phase:

an aqueous dispersion of film-forming polymer particles,

an aqueous microdispersion of particles of a first wax having a melting point of greater than or equal to 70° C., the particles of the first wax having an average size of less than 1 μm, and

an aqueous dispersion of particles of a second wax having a melting point of less than 70° C., the particles of the second wax having an average size of greater than or equal to 1 μm.

A subject of the invention is also a cosmetic process for making up or for non-therapeutic care of keratin materials, especially eyelashes, comprising the application to the keratin materials of a composition as defined above.

A subject of the invention is also the use of a composition as defined above to obtain a deposit, especially a make-up, on keratin materials, which is thick and which shows good staying power, and/or to curl keratin fibres, especially the eyelashes.

A subject of the invention is also the use

of an aqueous dispersion of film-forming polymer particles,

of an aqueous microdispersion of particles of a first wax having a melting point of greater than or equal to 70° C., the particles of the first wax having an average size of less than or equal to 1 μm, and

of an aqueous dispersion of particles of a second wax with a melting point of less than 70° C., the particles of the second wax having an average size of greater than or equal to 1 μm,

in a composition comprising a physiologically acceptable medium containing an aqueous phase, to obtain a deposit, especially a make-up, on keratin materials, which is thick and which shows good staying power, and/or to curl the keratin fibres, especially the eyelashes.

In addition, the composition according to the invention makes it possible to obtain a film deposited on keratin materials which is resistant to cold water and which is removable with warm water, that is to say with water at a temperature of greater than or equal to 35° C. (temperature measured at atmospheric pressure), and especially ranging from about 35° C. to 50° C. The make-up is removed very simply with warm water and in particular with warm water containing no detergent such as soaps.

The expression “physiologically acceptable” should be understood as meaning a medium which is compatible with keratin materials, for instance a cosmetic medium.

a) The Film-forming Polymer in Aqueous Dispersion:

The composition according to the invention contains a film-forming polymer which is in the form of particles in aqueous dispersion, generally known as latex or pseudolatex.

In the present application, the expression “film-forming polymer” means a polymer which is capable of forming, by itself or in the presence of a film-forming auxiliary agent, a continuous film which adheres to a support, especially to keratin materials, for instance the eyelashes.

Among the film-forming polymers which may be used in the composition of the present invention, mention may be made of synthetic polymers, of free-radical type or of polycondensate type, polymers of natural origin, and mixtures thereof.

The expression “free-radical film-forming polymer” means a polymer obtained by polymerization of monomers containing unsaturation, especially ethylenic unsaturation, each monomer being capable of homopolymerizing (unlike polycondensates).

The film-forming polymers of free-radical type may especially be vinyl polymers or copolymers, especially acrylic polymers.

The vinyl film-forming polymers may result from the polymerization of monomers containing ethylenic unsaturation having at least one acid group and/or esters of these acid monomers and/or amides of these acid monomers.

Monomers bearing an acid group which may be used include α,β-ethylenic unsaturated carboxylic acids such as acrylic acid, methacrylic acid, crotonic acid, maleic acid or itaconic acid. (Meth)acrylic acid and crotonic acid are preferably used, and more preferably (meth)acrylic acid.

The esters of acid monomers are advantageously chosen from the esters of (meth)acrylic acid (also known as (meth)acrylates), especially alkyl (meth)acrylates, in particular of a C₁-C₃₀ and preferably a C₁-C₂₀ alkyl, aryl (meth)acrylates, in particular of a C6-C₁₀ aryl, and hydroxyalkyl (meth)acrylates, in particular of a C₂-C₆ hydroxyalkyl.

Among the alkyl (meth)acrylates which may be mentioned are methyl methacrylate, ethyl methacrylate, butyl methacrylate, isobutyl methacrylate, 2-ethylhexyl methacrylate, lauryl methacrylate and cyclohexyl methacrylate.

Among the hydroxyalkyl (meth)acrylates which may be mentioned are hydroxyethyl acrylate, 2-hydroxypropyl acrylate, hydroxyethyl methacrylate and 2-hydroxypropyl methacrylate.

Among the aryl (meth)acrylates which may be mentioned are benzyl acrylate and phenyl acrylate.

The (meth)acrylic acid esters that are particularly preferred are the alkyl (meth)acrylates.

According to the present invention, the alkyl group of the esters may be either fluorinated or perfluorinated, that is to say that some or all of the hydrogen atoms of the alkyl group are replaced with fluorine atoms.

Amides of the acid monomers which may be mentioned, for example, are (meth)acrylamides, and especially N-alkyl(meth)acrylamides, in particular of a C₂-C₁₂ alkyl. Among the N-alkyl(meth)acrylamides which may be mentioned are N-ethylacrylamide, N-t-butylacrylamide, N-t-octylacrylamide and N-undecylacrylamide.

The vinyl film-forming polymers may also result from the homopolymerization or copolymerization of monomers chosen from vinyl esters and styrene monomers. In particular, these monomers may be polymerized with acid monomers and/or esters thereof and/or amides thereof, such as those mentioned above.

Examples of vinyl esters which may be mentioned are vinyl acetate, vinyl neodecanoate, vinyl pivalate, vinyl benzoate and vinyl t-butylbenzoate.

Styrene monomers which may be mentioned are styrene and α-methylstyrene.

The list of monomers given is not limiting, and it is possible to use any monomer known to those skilled in the art falling within the categories of acrylic and vinyl monomers (including monomers modified with a silicone chain).

As acrylic film-forming polymer which may be used according to the invention, mention may be made of those sold under the names Neocryl XK-90®, Neocryl A-1070®, Neocryl A-1090®, Neocryl BT-62®, Neocryl A-1079® and Neocryl A-523® by the company Avecia-Neoresins, Dow Latex 432® by the company Dow Chemical, and Daitosol 5000 AD by the company Daito Kasey Kogyo.

Among the film-forming polycondensates which may also be mentioned are polyurethanes, polyesters, polyesteramides, fatty-chain polyesters, polyamides and epoxyester resins. Polyurethanes are preferably used.

The polyurethanes may be chosen from anionic, cationic, nonionic and amphoteric polyurethanes, polyurethane-acrylics, polyurethane-polyvinyl-pyrrolidones, polyester-polyurethanes, polyether-polyurethanes, polyureas and polyurea-polyurethanes, and mixtures thereof.

The film-forming polyurethane may be, for example, an aliphatic, cycloaliphatic or aromatic polyurethane, polyurea-urethane or polyurea copolymer, comprising, alone or as a mixture:

at least one block of aliphatic and/or cycloaliphatic and/or aromatic polyester origin, and/or

at least one branched or non-branched silicone block, for example a dimethylsiloxane or polymethylphenyl-siloxane, and/or

at least one block comprising fluoro groups.

The film-forming polyurethanes as defined in the invention may also be obtained from branched or unbranched polyesters, or from alkyds comprising labile hydrogens which are modified by reaction with a diisocyanate and a difunctional (for example dihydro, diamino or hydroxyamino) organic compound, also comprising either a carboxylic acid or carboxylate group, or a sulphonic acid or sulphonate group, or alternatively a neutralizable tertiary amine group or a quaternary ammonium group.

As film-forming polyurethane which may be used according to the invention, it is possible to use those sold under the names Neorez R-981® and Neorez R-974® by the company Avecia-Neoresins, Avalure UR-405®, Avalure UR-410®, Avalure UR-425®, Avalure UR-450®, Sancure 875®, Sancure 861®, Sancure 878® and Sancure 2060® by the company Goodrich, and Impranil 85® by the company Bayer.

The polyesters can be obtained, in a known manner, by polycondensation of dicarboxylic acids with polyols, in particular diols.

The dicarboxylic acid can be aliphatic, alicyclic or aromatic. Examples of such acids which may be mentioned are: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norbornanedicarboxylic acid, diglycolic acid, thiodipropionic acid, 2,5-naphthalenedicarboxylic acid and 2,6-naphthalenedicarboxylic acid. These dicarboxylic acid monomers can be used alone or in a combination of at least two dicarboxylic acid monomers. Among these monomers, the ones preferably chosen are phthalic acid, isophthalic acid and terephthalic acid.

The diol can be chosen from aliphatic, alicyclic and aromatic diols. The diol preferably used is one chosen from: ethylene glycol, diethylene glycol, triethylene glycol, 1,3-propanediol, cyclohexanedimethanol, 1,4-butanediol. Other polyols which can be used are glycerol, pentaerythritol, sorbitol and trimethylolpropane.

The polyesteramides can be obtained in a similar manner to that for the polyesters, by polycondensation of diacids with diamines or amino alcohols. Diamines which can be used are ethylenediamine, hexamethylenediamine and meta- or para-phenylenediamine. An amino alcohol which can be used is monoethanolamine.

The polyester can also comprise at least one monomer bearing at least one group —SO₃M, with M representing a hydrogen atom, an ammonium ion NH₄⁺ or a metal ion such as, for example, an Na⁺, Li⁺, K⁺, Mg²⁺, Ca²⁺, Cu²⁺, Fe²⁺ or Fe ³⁺ ion. A difunctional aromatic monomer comprising such a group —SO₃M can be used in particular.

The aromatic nucleus of the bifunctional aromatic monomer also bearing a group —SO₃M as described above can be chosen, for example, from benzene, naphthalene, anthracene, biphenyl, oxybiphenyl, sulphonylbiphenyl and methylenebiphenyl nuclei. Examples of bifunctional aromatic monomers also bearing a group —SO₃M which may be mentioned are: sulphoisophthalic acid, sulphoterephthalic acid, sulphophthalic acid, 4-sulphonaphthalene-2,7-dicarboxylic acid.

Copolymers based on isophthalate/sulphoisophthalate, and more particularly copolymers obtained by condensation of diethylene glycol, cyclohexanedimethanol, isophthalic acid and sulphoisophthalic acid, are preferably used in the compositions which form the subject of the invention. Such polymers are sold, for example, under the brand name Eastman AQ by the company Eastman Chemical Products.

The polymers of natural origin, which are optionally modified, may be chosen from shellac resin, sandarac gum, dammar resins, elemi gums, copal resins and water-insoluble cellulose polymers, and mixtures thereof.

Mention may also be made of polymers resulting from the free-radical polymerization of one or more free-radical monomers inside and/or partially at the surface of pre-existing particles of at least one polymer chosen from the group consisting of polyurethanes, polyureas, polyesters, polyesteramides and/or alkyds. These polymers are generally known as hybrid polymers.

The dispersion comprising one or more film-forming polymers may be prepared by a person skilled in the art on the basis of his general knowledge.

The size of the polymer particles in aqueous dispersion may range from 10 nm to 500 nm and preferably from 20 nm to 300 nm.

Advantageously, a film-forming polymer with a water uptake of less than or equal to 50%, preferably less than or equal to 40%, more preferably less than or equal to 30% and better still less than or equal to 20% is used.

According to the present application, the expression “water uptake of the film-forming polymer” means the percentage of water absorbed by the polymer after immersing it for 10 minutes in water at 20° C. The water uptake is measured for a layer 300 μm thick (before drying) deposited on a plate and then dried for 24 hours at 30° C. and at 50% relative humidity; pieces of about 1 cm² cut out of the dry film are weighed (mass measurement M1) and then immersed in water for 10 minutes; after immersion, the piece of film is wiped to remove the excess surface water and then weighed (mass measurement M2). The difference M2-M1 corresponds to the amount of water absorbed by the polymer.

The water uptake is also equal to [(M2-M1)/M1]×100 and is expressed as a weight percentage of water relative to the weight of polymer.

The film-forming polymer in aqueous dispersion may be present in the composition according to the invention in a solids content ranging from 1% to 60% by weight relative to the total weight of the composition, preferably from 5% to 40% by weight and better still from 10% to 30% by weight.

The composition according to the invention may comprise a film-forming auxiliary agent for promoting the formation of a film with the particles of the film-forming polymer. Such a film-forming agent may be chosen from any compound known to those skilled in the art as being capable of fulfilling the desired function, and especially may be chosen from plasticizers and coalescers.

The aqueous phase of the composition may consist essentially of water. It may also comprise a mixture of water and of water-miscible solvent, for instance lower monoalcohols containing from 1 to 5 carbon atoms, such as ethanol or isopropanol, glycols containing from 2 to 8 carbon atoms, such as propylene glycol, ethylene glycol, 1,3-butylene glycol or dipropylene glycol, C₃-C₄ ketones and C₂-C₄ aldehydes. The aqueous phase (water and optionally the water-miscible organic solvent) represents, in practice, from 5% to 99.4% by weight relative to the total weight of the composition.

b) The First Wax:

The composition according to the invention moreover comprises an aqueous microdispersion of particles of a first wax having a melting point of greater than or equal to 70° C. The expression “aqueous microdispersion of wax” means an aqueous dispersion of wax particles, in which the average size of the said wax particles is less than or equal to about 1 μm.

The expression “average size of less than or equal to 1 μm” means the size at which at least 50% by volume of the particles are smaller than or equal to the average size of 1 μm. The size of the wax particles may be measured using a granulometer, for example that sold under the reference Mastersizer 2000 by the company Malvern.

In the present application, the wax is a lipophilic compound which is solid at ambient temperature (25° C.), which undergoes a reversible solid/liquid change of state, and which has a melting point of greater than or equal to 30° C. which may be up to 120° C. By bringing the wax to the liquid state (melting), it is possible to make it miscible with oils and to [lacuna]

The first wax may also have a hardness ranging from 0.05 MPa to 15 MPa. The hardness is determined by measuring the compressive force, measured at 20° C. using a texturometer sold under the name TA-TX2i by the company Rheo, equipped with a stainless steel cylinder 2 mm in diameter travelling at a measuring speed of 0.1 mm/s, and penetrating into the wax to a penetration depth of 0.3 mm. To carry out the hardness measurement, the wax is melted at a temperature equal to the melting point of the wax+20° C. The molten wax is poured into a container 30 mm in diameter and 20 mm deep. The wax is recrystallized at ambient temperature (25° C.) over 24 hours and the wax is then stored for at least 1 hour at 20° C. before carrying out the hardness measurement. The hardness value is the compressive force measured divided by the area of the texturometer cylinder in contact with the wax.

Examples of the first wax which may be used are carnauba wax, microcrystalline waxes, rice bran wax, Chinese insect waxes, shellac wax, montan wax, ouricurry wax, sugarcane wax, polyethylene waxes and hydrogenated castor oil, and mixtures thereof.

It is also possible to use commercial mixtures of self-emulsifying waxes containing a wax and surfactants. The wax sold under the name “Cire Auto Lustrante OFR” by Tiscco, which contains carnauba wax and paraffin wax combined with nonionic surfactants, or the self-emulsifying wax sold under the name “Cerax A.O. 28/B” by La Ceresine, which contains alfalfa wax combined with a nonionic surfactant, may be used, for example. These commercial mixtures make it possible to prepare wax microdispersions simply by adding water.

The wax microdispersions sold under the names “Aquacer 537” and “Aquacer 537” by the company Byk Cera may also be used.

The composition according to the invention may preferably comprise from 0.1% to 50% by weight of solids of the first wax, especially from 1% to 30% by weight, relative to the total weight of the composition, and preferably from 5% to 20% by weight.

The composition may also comprise an amount of surfactant which is sufficient to allow a wax microdispersion and a stable final composition to be obtained. It may especially comprise 0.01% to 5% by weight of common surfactant, which may be chosen from the following compounds:

anionic surfactants, especially optionally unsaturated fatty acid salts containing, for example, 12 to 18 carbon atoms; alkaline salts or salts of organic bases of alkylsulphuric and alkylsulphonic acids containing 12 to 18 carbon atoms or of alkylarylsulphonic acids in which the alkyl chain contains 6-18 carbon atoms; sulphate ethers;

nonionic surfactants, especially polyalkoxylated and/or polyglycerolated surfactants, and in particular fatty acids or fatty acid amides; fatty alcohols or alkylphenols; fatty acid esters of polyols; alkanediols and alkyl ethers of alkanediols. Mention may also be made of triglyceryl alkyl carbonates, oxyethylenated or propoxylated derivatives of lanolin alcohols, of lanolin fatty acids, or of mixtures thereof;

cationic surfactants, especially quaternary ammonium derivatives.

The wax or mixture of waxes may be combined with one or more fatty additives (oily and/or pasty). Mention may be made especially of plant oils, for instance sunflower oil or jojoba oil; mineral oils, for instance liquid paraffin; silicone oils; petroleum jelly or lanolin; fluoro oils; hydrocarbon-based oils containing a perfluoro group; fatty alkyl esters.

It is possible also to introduce into the microparticulate waxy phase liposoluble active ingredients, such as UV screening agents, liposoluble vitamins or liposoluble cosmetic active agents.

Advantageously, the first wax and the film-forming polymer in aqueous dispersion may be present in the composition in a film-forming polymer/first wax weight ratio ranging from 50/50 to 95/5 and better still ranging from 60/40 to 80/20.

c) The Second Wax:

The composition also comprises a second wax having a melting point of less than 70° C., in the form of particles with an average size of greater than or equal to 1 μm, preferably greater than or equal to 1.3 μm, dispersed in the aqueous phase. The average size of the particles of the second wax is different from and greater than the average size of the particles of the first wax. In particular, the average size of the particles of the second wax may range from 1 μm to 10 μm and preferably from 1.3 μm to 5 μm.

The expression “average size of greater than 1 μm” means the size at which at least 50% by volume of the particles are larger than the average size of 1 μm.

The second wax makes it possible to obtain a thick make-up result on the eyelashes; the mascara is then referred to as being loading.

The second wax preferably has a melting point of greater than or equal to 30° C. and less than 70° C.

The second wax may be present in a content ranging from 0.1% to 40% by weight relative to the total weight of the composition, preferably ranging from 2% to 30% and better still from 5% to 20% by weight.

The second wax may be chosen from beeswax, candelilla wax, paraffin wax, ozokerite, bayberry wax or myrtle wax, hydrogenated jojoba oil, palm butter, lanolin wax, sperm whale wax, Japan wax and sumach wax, ceresin, and mixtures thereof.

Advantageously, the film-forming polymer in aqueous dispersion and the second wax may be present in the composition in a film-forming polymer/second wax weight ratio ranging from 40/60 to 95/5 and preferably from 55/45 to 80/20.

d) The Additives:

The aqueous phase of the composition may also comprise an additional water-soluble film-forming polymer, which is especially present in a content ranging from 0.01% to 5% by weight relative to the total weight of the composition.

Water-soluble polymers which may be mentioned in particular include:

water-soluble cellulose polymers, for instance hydroxyethylcellulose, hydroxypropylcellulose, carboxymethylcellulose, hydroxypropylethylcellulose or ethylhydroxyethylcellulose;

keratin derivatives, such as keratin hydrolysates and sulphonic keratins;

anionic, cationic, amphoteric or nonionic chitin or chitosan derivatives, and especially hydroxypropyl chitosan;

cellulose derivatives such as hydroxyethylcellulose, hydroxypropylcellulose, methylcellulose, ethylhydroxyethylcellulose, carboxymethylcellulose and quaternized cellulose derivatives;

acrylic polymers or copolymers, such as polyacrylates or polymethacrylates;

polyvinyl alcohols and polyvinylpyrrolidones;

vinyl copolymers, such as copolymers of methyl vinyl ether and of malic anhydride, or the copolymer of vinyl acetate and of crotonic acid;

polyethylene glycols;

polymers of natural origin, which are optionally modified, such as:

arabic gums, guar gum, xanthan derivatives or karaya gum;

alginates and carrageenates;

glycoaminoglycans, and hyaluronic acid and its derivatives;

shellac resin, sandarac gum, dammar resins, elemi gums and copal resins;

deoxyribonucleic acid.

The composition may also comprise at least one dyestuff, for instance pulverulent compounds, for example in a proportion of from 0.01% to 50% of the total weight of the composition. The pulverulent compounds may be chosen from the pigments and/or nacres usually used in cosmetic compositions. Advantageously, the pulverulent compounds represent from 0.1% to 25% of the total weight of the composition and better still from 1% to 20%.

The pigments may be white or coloured, and mineral and/or organic. Among the mineral pigments which may be mentioned are titanium dioxide, optionally surface-treated, zirconium oxide or cerium oxide, and iron oxide or chromium oxide, manganese violet, ultramarine blue, chromium hydrate and ferric blue. Among the organic pigments which may be mentioned are carbon black, pigments of D & C type, and lacquers based on cochineal carmine or on barium, strontium, calcium or aluminium.

The nacreous pigments may be chosen from white nacreous pigments such as mica coated with titanium or with bismuth oxychloride, coloured nacreous pigments such as titanium mica with iron oxides, titanium mica with, especially, ferric blue or chromium oxide, titanium mica with an organic pigment of the abovementioned type, and nacreous pigments based on bismuth oxychloride.

The composition may also comprise fillers which may be chosen from those that are well known to those skilled in the art and which are commonly used in cosmetic compositions. The fillers may be mineral or organic, and lamellar or spherical. Mention may be made of talc, mica, silica, kaolin, Nylon powder (Orgasol from Atochem), poly-β-alanine powder and polyethylene powder, Teflon, lauroyllysine, starch, boron nitride, tetrafluoroethylene polymer powders, hollow microspheres such as Expancel (Nobel Industrie), Polytrap (Dow Corning), silicone resin microbeads (Tospearls from Toshiba, for example), precipitated calcium carbonate, magnesium carbonate and hydrocarbonate, hydroxyapatite, hollow silica microspheres (Silica Beads from Maprecos), glass or ceramic microcapsules, metal soaps derived from organic carboxylic acids containing from 8 to 22 carbon atoms and preferably from 12 to 18 carbon atoms, for example zinc, magnesium or lithium stearate, zinc laurate or magnesium myristate.

The composition according to the invention may also contain ingredients that are commonly used in cosmetics, such as trace elements, softeners, sequestering agents, fragrances, oils, silicones, thickeners, vitamins, proteins, ceramides, plasticizers, coalescers and cohesion agents, and also the acidifying or basifying agents usually used in cosmetics, emollients and preserving agents.

Needless to say, a person skilled in the art will take care to select this or these optional additional compounds, and/or the amount thereof, such that the advantageous properties of the composition according to the invention are not, or are not substantially, adversely affected by the envisaged addition.

The composition according to the invention may be prepared according to the usual methods of the fields under consideration. It may be prepared especially by mixing, in a first stage, the polymer in aqueous dispersion with the non-wax constituents of the aqueous phase, and then heating this mixture to a temperature ranging from 30° C. to 80° C., in particular to a temperature slightly above the melting point of the second wax. Next, the second wax is melted and is added to the heated aqueous dispersion, with stirring, which gives particles of the second wax having a size of greater than or equal to 1 μm. The mixture is then cooled to room temperature (about 25° C.), followed by addition of the micro-dispersion of the first wax prepared according to the preparation method described previously. The other ingredients, for instance the dyestuffs and the preserving agents, are then added.

The invention is illustrated in greater detail in the examples which follow.

### EXAMPLE 1

A microdispersion of carnauba wax having the composition below was prepared:

| | | |
|---|---|---|
| Carnauba wax | | 27 g |
| Polyoxyethylenated (30 EO) glyceryl | | 6.75 g |
| monostearate (Tagat S from Goldschmidt) | | |
| Ethanol | | 10 g |
| Water | qs | 100 g |

The wax and the surfactant were heated to 90° C. while homogenizing the mixture with moderate stirring. Next, the water heated to 90° C. was incorporated with continued stirring. The mixture was cooled to ambient temperature and the ethanol was added to obtain a wax microdispersion with a mean particle diameter of about 170 nm.

### EXAMPLE 2

A mascara having the composition below was prepared:

| | | |
|---|---|---|
| Polyurethane as an aqueous dispersion, | | 14 g AM |
| sold under the name Avalure UR 425 by | | |
| the company Goodrich, at 49% by weight | | |
| of active materials | | |
| Wax microdispersion of Example 1 | | 31.5 g |
| Beeswax | | 10 g |
| Thickener | | 1.9 g |
| Ethanol | | 7 g |
| Propylylene glycol | | 5 g |
| Pigments | | 5 g |
| Preserving agents | qs | |
| Water | qs | 100 g |

This mascara was prepared by mixing together the polyurethane in aqueous dispersion and the thickener, and heating this mixture to 65° C. Next, the beeswax was melted to disperse it with stirring in the thickened latex so as to obtain particles of beeswax having an average size of greater than 1 μm. The mixture was then cooled to room temperature (about 25° C.), followed by addition of the ground pigments in propylene glycol and the wax microdispersion, and also the preserving agents dissolved in ethanol.

The mascara applies easily to the eyelashes and forms a loading make-up which is resistant to cold water. It also gives good curling of the eyelashes. The mascara is easy to remove with warm (40° C.) water.

### EXAMPLE 3

A mascara having the composition below was prepared:

| | | |
|---|---|---|
| Polyurethane as an aqueous dispersion, sold | | 16 g AM |
| under the name Avalure UR 425 by the company | | |
| Goodrich, at 49% by weight of active materials | | |
| Microdispersion of a mixture of acrylic | | 31.5 g |
| copolymer wax and of mineral wax (at 20% by | | |
| weight of wax), sold under the name “Aquacer | | |
| 520” by the company Byk Cera | | |
| Candelilla wax | | 15 g |
| Thickener | | 1.9 g |
| Ethanol | | 7 g |
| Propylene glycol | | 5 g |
| Pigments | | 5 g |
| Preserving agents | qs | |
| Water | qs | 100 g |

The mascara applies easily to the eyelashes and forms a loading make-up, which is resistant to cold water (20° C.). It also gives good curling of the eyelashes.

1. Composition comprising, in a physiologically acceptable medium containing an aqueous phase:
an aqueous dispersion of film-forming polymer particles,
an aqueous microdispersion of particles of a first wax having a melting point of greater than or equal to 70° C., the particles of the first wax having an average size of less than 1 μm, and
an aqueous dispersion of particles of a second wax having a melting point of less than 70° C., the particles of the second wax having an average size of greater than or equal to 1 μm.

2. Composition according to claim 1, characterized in that the film-forming polymer particles have a size ranging from 10 to 500 nm and preferably ranging from 20 to 300 nm.

3. Composition according to either of claims 1 and 2, characterized in that the film-forming polymer in aqueous dispersion is chosen from free-radical polymers, polycondensates and polymers of natural origin.

4. Composition according to any one of the preceding claims, characterized in that the film-forming polymer in aqueous dispersion is chosen from the group formed by vinyl polymers, polyurethanes, polyesters and polyamides.

5. Composition according to any one of the preceding claims, characterized in that the film-forming polymer in aqueous dispersion is a polyurethane.

6. Composition according to any one of the preceding claims, characterized in that the film-forming polymer has a water uptake of less than or equal to 50%, preferably less than or equal to 40% and better still less than or equal to 30%.

7. Composition according to any one of the preceding claims, characterized in that the film-forming polymer in aqueous dispersion is present in a content ranging from 1% to 60% by weight relative to the total weight of the composition, preferably ranging from 5% to 40% by weight and better still from 10% to 30% by weight.

8. Composition according to any one of the preceding claims, characterized in that the first wax has a melting point ranging from 70° C. to 120° C.

9. Composition according to any one of the preceding claims, characterized in that the microdispersion of the first wax comprises wax particles with an average size of less than 1 μm and preferably less than 0.5 μm.

10. Composition according to any one of the preceding claims, characterized in that the first wax is chosen from the group formed by carnauba wax, microcrystalline waxes, rice bran wax, Chinese insect waxes, shellac wax, montan wax, ouricurry wax, sugarcane wax, polyethylene waxes and hydrogenated castor oil, and mixtures thereof.

11. Composition according to any one of the preceding claims, characterized in that the first wax has a hardness ranging from 0.05 MPa to 15 MPa and preferably ranging from 6 MPa to 15 MPa.

12. Composition according to any one of the preceding claims, characterized in that the first wax of the said microdispersion is present in a solids content ranging from 0.1% to 50% by weight relative to the total weight of the composition, preferably ranging from 1% to 30% by weight and better still ranging from 5% to 20% by weight.

13. Composition according to any one of the preceding claims, characterized in that the film-forming polymer in aqueous dispersion and the first wax are present in the composition in a film-forming polymer/first wax weight ratio ranging from 50/50 to 95/5 and preferably from 60/40 to 80/20.

14. Composition according to any one of the preceding claims, characterized in that the particles of the microdispersion of the first wax also comprise oily and/or pasty fatty additives and/or a liposoluble additive/active agent.

15. Composition according to one of the preceding claims, characterized in that the composition also comprises at least one surfactant.

16. Composition according to any one of the preceding claims, characterized in that the particles of the second wax have a size of greater than or equal to 1.3 μm.

17. Cosmetic composition according to any one of the preceding claims, characterized in that the second wax has a melting point of greater than or equal to 30° C. and less than 70° C.

18. Composition according to any one of the preceding claims, characterized in that the second wax is chosen from the group formed by beeswax, candelilla wax, paraffin wax, ozokerite, bayberry wax or myrtle wax, hydrogenated jojoba oil, palm butter, lanolin wax, sperm whale wax, Japan wax, sumach wax and ceresin, and mixtures thereof.

19. Composition according to any one of the preceding claims, characterized in that the second wax is present in a content ranging from 0.1% to 30% by weight relative to the total weight of the composition, preferably ranging from 1% to 25% and better still from 5% to 20% by weight.

20. Composition according to any one of the preceding claims, characterized in that the film-forming polymer in aqueous dispersion and the second wax are present in the composition in a film-forming polymer/second wax weight ratio ranging from 40/60 to 95/5 and preferably from 55/45 to 80/20.

21. Composition according to any one of the preceding claims, characterized in that the composition also comprises an additional water-soluble film-forming polymer.

22. Composition according to any one of the preceding claims, characterized in that the composition also comprises an additive chosen from the group formed by pigments, nacres, fillers, trace elements, softeners, sequestering agents, fragrances, oils, silicones, thickeners, vitamins, proteins, ceramides, plasticizers, coalescers, cohesion agents, basifying agents, acidifying agents, emollients and preserving agents.

23. Composition according to any one of the preceding claims, characterized in that it is in the form of a mascara.

24. Mascara comprising, in a physiologically acceptable medium containing an aqueous phase,:
an aqueous dispersion of film-forming polymer particles,
an aqueous microdispersion of particles of a first wax having a melting point of greater than or equal to 70° C., the particles of the first wax having an average size of less than 1 μm, and
an aqueous dispersion of particles of a second wax having a melting point of less than 70° C., the particles of the second wax having an average size of greater than or equal to 1 μm.

25. Process for making up or for non-therapeutic care of keratin fibres, especially the eyelashes, characterized in that it comprises the application to the keratin fibres of a composition according to any one of claims 1 to 23.

26. Use of a composition according to any one of claims 1 to 24, to obtain a deposit, especially a make-up, on keratin materials, which is thick and which has good staying power, and/or to curl the keratin fibres, especially the eyelashes.

27. Use of a composition according to any one of claims 1 to 24, to obtain a film deposited on the keratin materials which is resistant to cold water and removable with warm water.

28. Use
of an aqueous dispersion of film-forming polymer particles,
of an aqueous microdispersion of particles of a first wax having a melting point of greater than or equal to 70° C., the particles of the first wax having an average size of less than or equal to 1 μm, and
of an aqueous dispersion of particles of a second wax having a melting point of less than 70° C., the particles of the second wax having an average size of greater than or equal to 1 μm,
in a composition comprising a physiologically acceptable medium containing an aqueous phase, to obtain a deposit on keratin materials which is thick and which has good staying power, and/or to curl the keratin fibres, especially the eyelashes.

29. Use
of an aqueous dispersion of film-forming polymer particles,
of an aqueous microdispersion of particles of a first wax having a melting point of greater than or equal to 70° C., the particles of the first wax having an average size of less than or equal to 1 μm, and
of an aqueous dispersion of particles of a second wax having a melting point of less than 70° C., the particles of the second wax having an average size of greater than or equal to 1 μm,
in a composition comprising a physiologically acceptable medium containing an aqueous phase, to obtain a film deposited on the keratin materials which is resistant to cold water and which is removable with warm water.
